(19) United States
(12) Patent Application Publication
(10) Pub. No.: US 2021/0361401 A1
(43) Pub. Date: Nov. 25, 2021

(54) ANAESTHETIC VAPORIZER FILLER

(71) Applicants: Colin DUNLOP, East Ryde, NSW (AU); James DUNLOP, Gladesville, NSW (AU)

(72) Inventors: Colin DUNLOP, East Ryde, NSW (AU); James DUNLOP, Gladesville, NSW (AU)

(21) Appl. No.: 17/255,930

(22) PCT Filed: Jan. 16, 2020
(86) PCT No.: PCT/AU2020/000007

§ 371 Date: Dec. 23, 2020

(30) Foreign Application Priority Data
Jan. 18, 2019 (AU) 2019900152

(51) Int. Cl. A61D 7/04

(52) U.S. Cl. CPC A61D 7/04

(57) ABSTRACT

A filler for an anaesthetic vaporizer used for veterinary surgery. The filler is attached to a cylindrical bucket portion of the vaporizer. The filler is curved in shape and having a concave mounting surface that is obverse to the cylindrical bucket portion. The filler having fluid conduits therein interconnecting first apertures on the concave mounting concave surface and a filler-port unit, the first apertures on the mounting surface aligning with associated second apertures on said bucket. A gasket with a plurality of first holes aligned with first and second apertures is disposed between the bucket and the filler.

### TECHNICAL FIELD

This invention relates to an anaesthetic vaporizer filler. In particular, the present invention is described with reference to an anaesthetic vaporizer filler for use in veterinary surgery.

### BACKGROUND

An anaesthetic vaporizer is a device used to convert a liquid anaesthetic into a breathable form (vapour) and deliver the anaesthetic vapour in specific concentrations so that controlled doses can be administered to the patient. Many variables affect the concentration produced, to including the type and temperature of the liquid anaesthetic, the pressures in the anaesthesia system delivering the vapour, and the type and flow rate of the “carrier gases” used to produce the vapour. The carrier gas can be oxygen or a mixture of gases such as oxygen, nitrous oxide, room air, and re-breathed air.

Anaesthetic vaporizers ensure effective and safe addition of inhalant anaesthetics to the fresh is gas flow exiting the common gas outlet of an anaesthetic machine. Typically, the inhalant anaesthetics are any of halothane, isoflurane, sevoflurane and desflurane.

Modern inhalant anaesthetics are liquids at room temperature and so must be converted to gas prior to delivery to an animal patient, via the process of vaporisation. During vaporisation, molecules of anaesthetic on the surface of the liquid phase escape and become vapour (gas).

This process continues until the pressure exerted by the molecules in the vapour phase is maximal (e.g. equilibrium is reached between the liquid and gas phase)—this pressure is the “saturated vapour pressure” (SVP). The SVP is different for each anaesthetic agent, and differs according to temperature. It is the pressure of anaesthetic gas in a gas mixture (its “partial pressure”) that determines the “dose” that will reach the brain of the animal that is breathing the gas mixture. The SVP of the inhalant anaesthetics that are used in veterinary anaesthesia far exceeds the dose required to produce anaesthesia, so vaporizers must be used to ensure that a small and precise concentration of anaesthetic gas is added to the fresh gas flow (e.g. oxygen).

The most important function of a vaporizer is to ensure that a precise and constant amount of anaesthetic gas is added to the final gas mixture that exits the common gas outlet of the anaesthetic machine. In order to do this the vaporizer must account for any changes in fresh gas flow, or temperature as these will affect the amount of anaesthetic liquid that is vaporised.

In veterinary surgery the widely available vaporizer is a plenum vaporizer, commonly known as an “out of circuit” vaporizer, driven by positive pressure from an anaesthetic machine. The performance of such plenum vaporizers depends extensively on the SVP of the volatile agent. This is unique to each agent, so it follows that each agent must only be used in its own specific vaporizer. Several safety systems have been devised so that filling a plenum vaporizer with the wrong agent is extremely difficult. One such safety system is the use of key-fills to ensure the correct bottles of liquid anaesthetic are connected to vaporizers designed and tuned for such anaesthetic.

SVP for any one agent varies with temperature, and plenum vaporizers are designed to operate within a specific temperature range. They have several features designed to compensate for temperature changes (especially cooling by evaporation). They often have a metal jacket, which equilibrates with the temperature in the room and provides a source of heat. In addition, the entrance to the vaporising chamber is controlled by a bimetallic strip, which admits more gas to the chamber as it cools, to compensate for the loss of efficiency of evaporation.

Filling anaesthetic plenum vaporizers with liquid anaesthetic has been a challenge when attempting to minimize spillage of volatile liquid anaesthetic, and risk to the user. This is due to anaesthetic inhalants being volatile liquids, with a low boiling point, and they tend to turn into vapour and increase pressure when excited by moving the bottle. This vapour will remain at the partial pressure of the anaesthetic being used inside the bottle and the vaporizer liquid chamber when the vaporizer is closed (usually in between 150 and 250 mmHg).

In earlier days, filling a vaporizer involved opening a side port on the reservoir or the entire unit, and pouring the volatile liquid directly from the bottle into a funnel. This was a delicate process which needed to be improved to address basic occupational health and safety.

Key-fill devices came into use in the 1970s. They rely on the vaporizer being set at ‘off’, meaning the chamber being filled and the bottle arc scaled and at equal pressure. In older models of vaporizer with no valve and no breather hole, this proved to be precarious and could cause a large amount of liquid anaesthetic to be vented from the filler when the bottle conduit is withdrawn from the fill port as the vaporizer reservoir depressurizes. This causes liquid anaesthetic to be ejected onto the user.

To overcome the problem of vapour lock in the system due to rising pressure when the vaporizer is being filled, a valve is usually added to seal the larger vaporizer reservoir between the bottle and filler. This allows the smaller filler to be primed before filling, and it was hoped that the vaporizer chamber would stay at the higher pressure and the excess anaesthetic in the smaller bottle and filler could be depressurized in a controlled manner. Small amounts of anaesthetic liquid however will in most instances still depressurize over the user when removing the filler.

More recently a breather hole has since been added to modern prior art vaporizer fill ports such that the entire system is open to atmosphere while filling, which lets out excess pressure in a controlled manner. This means that no liquid anaesthetic depressurizes over the user. However due to poor design placements of this breather hole, they do not really work well, so vaporizers were still difficult to fill, and liquid could still be ejected onto the user.

Fillers have also been problematic in selecting appropriate sealing material in the valve seat, valve stem and other components. This is because a veterinarian will typically need to fill a vaporizer at least once per week, putting high cyclic load on the valve seals. The environment of anaesthetics in liquid form has only been suitable for only a few materials without some form of chemical reaction. Standard 304 stainless steel screws have shown to corrode chemically in liquid anaesthetic due to solubility of anaesthetic.

One of the most widely available vaporizers ever made was the Cyprane Tec 3, utilising a cylindrical bucket (outer housing), and available initially as a funnel fill and then as a key-fill unit. A “prior art” key-fill Tec 3 vaporizer 101 is shown in FIG. 1, having a filler 102 with a conventional filler-port (filling lock mechanism) 103 mounted thereon, for receiving the key-fill adaptor used with liquid anaesthetic bottles.

Whilst such a Tec 3 vaporizer 101 works extremely well when serviced and maintained properly, it is difficult to fill and drain without the abovementioned issues presenting themselves. Filler 102 whilst disposed externally of bucket 104 has the problem that drain 105 sits below the bottom rim of bucket 104 as it is a gravity feed system, and it is fastened from the inside of the reservoir (sump) making it prone to corrosion issues. Because drain 105 extends below bucket 104, this means vaporizer 101 is not able to be placed upright on a level surface causing numerous issues during maintenance, removal and replacement, including occupational health and safety issues.

The abovementioned corrosion issues are at their worst when halothane is the anaesthetic. Halothane is the most corrosive anaesthetic used today and is still used in equine anaesthesia and in underdeveloped countries due to its low cost.

Another problem with the prior art filler 102, is that it has a square/rectangular edge configuration, and this with the various protruding components thereon, makes it more likely to have gas conduits and the like to catch or snag thereon when in use, adding a further complication to occupational health and safety issues.

As such the prior art vaporizers have posed serious leakage and spillage problems associated with filling them with liquid anaesthetic. mainly due to the design and attachment of the filler. Those fillers attached from inside the reservoir have additional occupational health and safety issues, as the filler can only be removed by disassembly of the vaporizer, which requires additional safety precautions, such as disassembly in a fume hood, adding considerably time and expense to service.

Furthermore, the filler gauges (sight glass level indicators) in prior art vaporizers have been quite small, making them difficult to see during filling, and related to the limited capacity of anaesthetic that can be introduced into the vaporizer.

Most of the prior art vaporizers made for veterinary use in recent times have used fillers that have requiring expensive six-axis CNC machines for manufacture, with considerable wastage of materials and significant plugging.

The present invention is to provide a filler for an anaesthetic vaporizer used for veterinary surgery that overcomes at least one of the problems associated with the prior art.

### SUMMARY OF INVENTION

In a first aspect the present invention consists of a filler for an anaesthetic vaporizer used for veterinary surgery, said filler attached to a cylindrical bucket portion of said vaporizer, wherein said filler is curved in shape and having a concave mounting surface that is obverse to said cylindrical bucket portion, said filler having fluid conduits therein interconnecting first apertures on said concave mounting surface and a filler-port unit, said first apertures on said concave mounting surface aligning with associated second apertures on said bucket, and a gasket with a plurality of first holes aligned with first and second apertures is disposed between said bucket and said filler.

Preferably said filler is provided with a plurality of third apertures, aligned with a plurality of fourth apertures in said bucket and a plurality of second holes in said gasket, said third plurality of apertures allowing for threaded fasteners to be inserted from external of said filler through said plurality of second holes in said gasket and through to said fourth apertures in said bucket to attach said filler to said bucket.

Preferably said threaded fasteners are captive screws that do not extend into the reservoir disposed within said cylindrical bucket.

Preferably said filler has a sight window disposed thereon at or near one end of said filler, and said opposed end has an upper portion overhanging a side mounting surface to which a filler port unit is attached, said filler port unit having a lower surface that is substantially flush with the bottom surface of said filler.

Preferably the fluidally connected breather holes in said filler are spaced both vertically and horizontally to each other.

Preferably the body of said filler is made of brass and coated with nickel.

Preferably said vaporizer is for vaporising anaesthetic liquid being dispensed to an animal patient via an associated anaesthetic delivery machine.

In a second aspect the present invention is a vaporizer and filler in combination, said vaporizer having a cylindrical outer bucket containing a reservoir for containing anaesthetic, said filler having a concave mounting surface obverse to said outer cylindrical bucket, said filler mounted to said vaporizer via said concave mounting surface abutting against said cylindrical outer bucket with a gasket disposed there between, said filler attached to said vaporizer by captive screws fitted from external of said vaporizer.

Preferably said filler has a filler-port unit attached thereto at or near a lower corner portion of said filler, and said filler-port unit having a lower surface that is substantially flush with the bottom surface of said filler.

Preferably said filler has fluidally connected breather holes therein spaced both vertically and horizontally to each other.

### BRIEF DESCRIPTION OF DRAWINGS

FIG. 1 is a perspective view of a “prior art” Tec 3 vaporizer with filler.

FIG. 2 is a perspective view of a vaporizer and filler in accordance with a first embodiment of the present invention.

FIG. 3 is an enlarged perspective view of the body of the filler shown in FIG. 1 with the valve and fill-port unit removed.

FIG. 4 is an enlarged perspective view of a gasket used with vaporizer and filler shown in FIG. 1.

FIG. 5 is a schematic of the rear view (concave surface) of the body of the filler shown in FIG. 3.

FIG. 6 is a schematic top view of the body of the filler shown in FIG. 3.

FIG. 7 is a schematic cross-sectional view of the body of the filler through G-G on FIG. 6.

### BEST MODE OF CARRYING OUT THE INVENTION

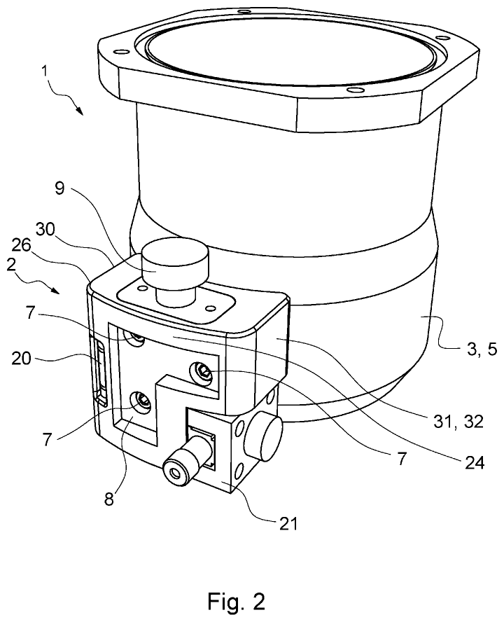

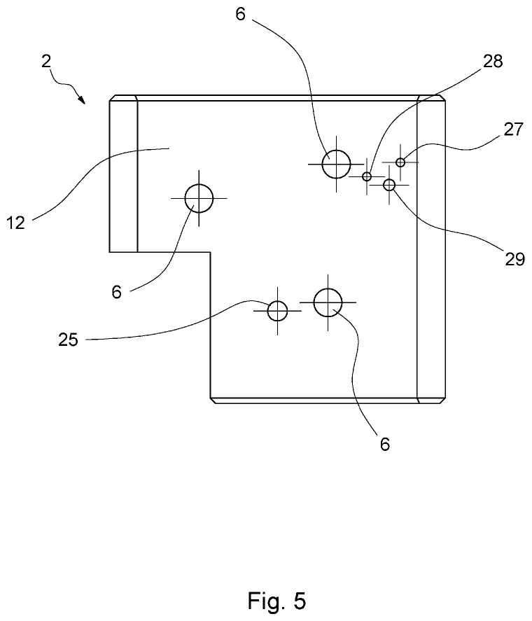

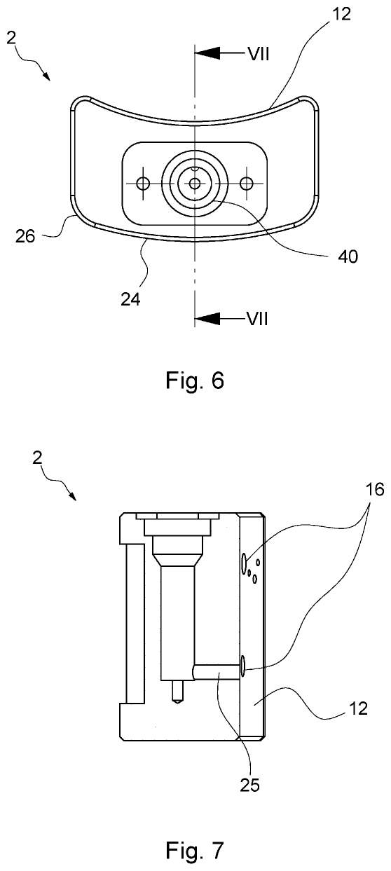

FIGS. 2 to 7 depict a first embodiment of a vaporizer 1 and a filler 2 attached thereto. Vaporizer 1 is preferably an anaesthetic vaporizer used with anaesthetic machine for veterinary patients.

External bucket 3 and the internal reservoir (not shown) disposed therein of vaporizer 1 are made of aluminium that is nickel coated by electroless process for heavy corrosion resistance, and then chrome finished for aesthetic purposes. The nickel coating material delivered by the electroless process is impervious to modem anaesthetics such as halothane and isoflurane and penetrates deeper bores over older conventional plating.

The body of filler 2 is preferably made of solid brass and nickel coated and chrome finished for aesthetic purposes.

In manufacture the single block of brass used to manufacture the body of filler 2, can have the various holes/bores machined on an inexpensive three-axis CNC machine. Filler 2 is curved entirely in one plane and has a concave mounting surface 12 that is substantially obverse to the outer surface 5 of cylindrical bucket 3. Concave mounting surface 12 of filler 2 abuts against the outer surface 5 of bucket 3 via a thin sheet gasket (seal) 4.

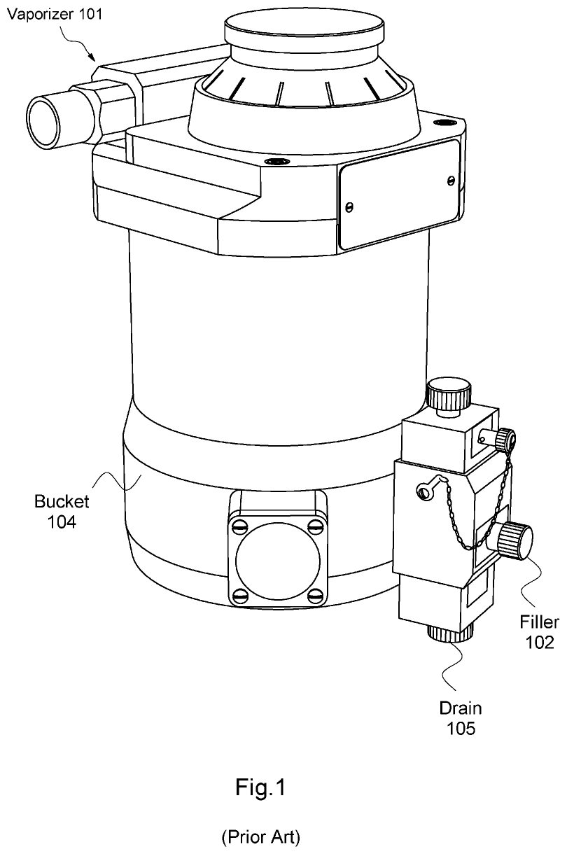

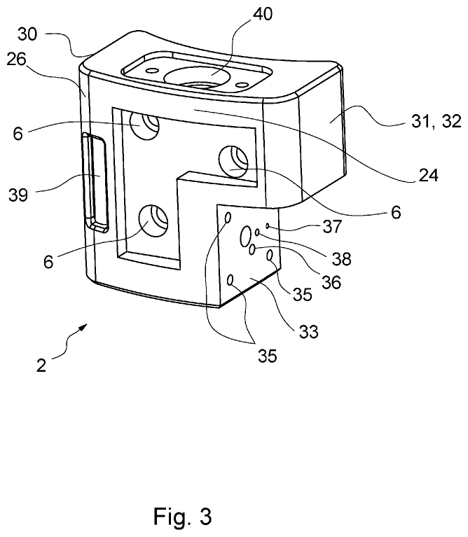

As can be seen in FIG. 3, the width of the body of filler 2 at its top end is about the same as its height dimension, which is significantly different to the prior art filler 102 as shown in FIG. 1, which looks significantly elongate with its height dimension significantly greater than its width. Furthermore, the depth of filler 2, namely the dimension to which it extends outwardly from bucket 3, is smaller than that of prior art 102. This configuration of filler 2, possible as result of concave mounting surface 12 has significant advantages which will be detailed later on.

Filler 2 has three apertures 6, through which three attachment screws 7 pass through to vaporizer 1 via gasket 4. Screws (captive screws) 7 are accessible from the external side of filler 2, and the three apertures 6 are disposed in an L-shaped recess 8, that can be covered by a sticker once filler 2 is attached to vaporizer 1.

Filler 2 has a fluid level sight-glass 20 disposed at one end 30 thereof where an elongate void 39 is disposed. On the opposite end 31, an upper portion 32 is disposed over (overhangs) a conventional filler-port unit (filling lock mechanism) 21 that is mounted to a set-back flat mounting surface 33. Filler-port unit 21 is a conventional device similar to that employed on the prior art Cyprane Tec 3 and is configured to receive a key-fill adaptor used with liquid anaesthetic bottles. Filler-port unit 21 when attached to mounting surface 33 does not extend below the bottom of the body of filler 2 and bucket 3, and preferably substantially flush therewith. This means that vaporizer 1 can be placed on a flat surface upright, making maintenance, removal and replacement straightforward.

Mounting surface 33 has attachment apertures 35 for mounting filler-port unit 21, fill aperture 36, breather aperture 37 and air breather return aperture 38. Breather return apertures 37, 38 are fluidally connected to breather hole 27, air return hole 28 and sight glass breather hole 29 shown on concave surface 12 of filler 2. Breather aperture 37 and air return 38 near the bottom of filler 2 are spaced apart from the associated fluidally connected breather hole 27 and air return hole 28 horizontally, and additionally they are disposed near opposite ends of filler 2. This spacing both horizontally and vertically between breather hole 27 and air return hole 28 and their respective associated breather aperture 37 and air return 38 means that the breather conduit connecting them within filler 2 is of greater length and therefore greater internal surface area. This means that should there be a droplet of liquid anaesthetic therein it has greater area to spread and there is less of a chance of spillage of anaesthetic during handling of vaporizer 1. This spacing of breather holes is possible because of the dimensional configuration, where the top end width of filler 2 is about the same as the height dimension, and as a result of the concave mounting surface 12 that allows for this width of filler 2 to mount to the cylindrical outer surface 5 of bucket 3.

Also, the significant vertical spacing between breather hole 27 and aperture 37 means that it is impossible to overfill vaporizer 1. This significantly reduces the risk of exposure of the anaesthetic to a user when filling and draining vaporizer 1.

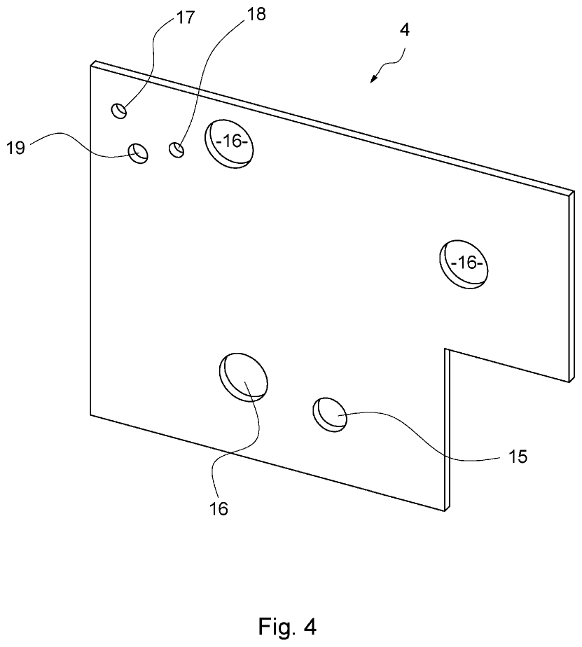

Gasket (seal) 4 is of thin sheet of PTFE material. As shown in FIG. 4, gasket 4 has three holes 16 that align with apertures 6 in filler 2. The three smaller holes near the uppermost hole 16 are gasket breather hole 17, gasket air return hole 18 and sight glass breather hole 19, which align with the breather hole 27, air return hole 28 and sight glass breather hole 29 of filler 2. Hole 15 of gasket 5 aligns with fill hole 25 on concave surface 12 of filler 2. In this embodiment gasket 4 is a rectangularly shaped and of constant thickness. However, in another not shown embodiment the gasket may have a narrow stepped peripheral border of greater thickness than the remaining central portion of the gasket.

Filler 1 is provided with a valve 9 for filling and draining, that is fitted to upper opening 40 of filler 2. A corrosion resistant spring-loaded PTFE nub seal (not shown), namely a dynamic valve stem fill/drain seal, is disposed on the shaft (not shown) of valve 9. This is a long-life seal with an expected service life of up to fifteen years.

Seals (not shown) for the fluid level sight glass 20 are three PTFE O-rings which are highly resistant to anaesthetic and have been proven to last more than five years if inserted and seated correctly. Fluid sight glass 20 is significant taller than in prior art arrangements, because there is a significant vertical height difference of the breather holes. This also results with a higher capacity tank for liquid anaesthetic. Filling vaporizer 1 is therefore easier as the user can easily watch the sight glass (fill gauge) 20 and the bottle in the same field of view. This is an improvement over the prior art window (sight glass) systems.

Preferably the body of filler 2 is machined to have a curved outer surface 24 and curved corners 26. The curved outer surface 24, curved corners 26 together with the mount location of filler-port unit 20, as well as the depth (the amount it projects from bucket 3) minimises the catch or snag potential when compared to prior art vaporizer/filler units.

An important feature of the present embodiment is that filler 2 is secured from the outside of the internal reservoir of vaporizer 1 using captive screws 7. As such the captive screws 7 are substantially clear of anaesthetic corrosion. Because captive screws 7 are removable from an external location without disassembly of the internal components of vaporizer 1, this makes servicing of vaporizer 1 and filler 2 easier and without less risk to the technician, than that posed by the prior art units.

In summary, as a result of the concave mounting surface 12 of filler 2 the following features are possible and advantageous.
Firstly, filler 2 is manufactured in a size where its top end width is about the same size as its height (see FIG. 3), and mountable to cylindrical outer surface 5;
Secondly, filler 2 has sight window 20 disposed thereon at one end of filler 2, and said opposed end has an upper portion overhanging a side mounting surface 33 to which filler port unit 21 is attached, and filler port unit 21 having a lower surface that is substantially flush with the bottom surface of filler 2. As such vaporizer 1 can be stood on a flat surface unlike the prior art standard Tec 3 model.
increased fluid level and reservoir capacity and the size of sight glass 20, as a result of breather hole spacing (vertically and horizontally), which provides less chance for spillage and better visual in the sight glass 20 for accurate filling

Further advantageous features of the present embodiment are as follows:
filler 2 as a result of external screws 7 accessible from external location, can be removed from vaporizer 1 without disassembly of the latter;
the spring-loaded valve shaft seal of valve 9 would require less servicing and last two to three times longer than prior valve seals. For example, it would be serviceable after ten years instead of two-five years of the prior art;
whilst valve seat is serviceable it will not be required for the life of vaporizer 1;
: and
no outer sharp corners, as a result of curved corners 26 minimising catch and snag points.

The embodiment of the filler 2 shown above utilises filler-port unit 21 to receive a key-fill adaptor used with liquid anaesthetic bottles as commonly used in the United Kingdom and Australia. However, it should be understood that in another not shown embodiment, the filler of the present invention, whilst still maintaining a curved mounting surface 12 in a similar fashion to filler 2 and mountable on bucket 3, could instead of filler port unit 21, use a not shown funnel-fill port (as preferred in the United States of America) located on the upper surface of the filler, at the location where valve 9 and upper opening 40 are disposed on earlier mentioned filler 2. In such not shown embodiment, the “overhang” configuration as provided in filler 2 for the filler port unit 21 is not required. As such in this not shown “filler” embodiment, the top and bottom widths would preferably be the same. However, as in the earlier embodiment, this not shown filler would not extend below the bottom of bucket 3, and preferably is substantially flush therewith, meaning vaporizer 1 to which it is attached can be placed on a flat surface upright, making maintenance, removal and replacement straightforward.

The terms “comprising” and “including” (and their grammatical variations) as used herein are used in an inclusive sense and not in the exclusive sense of “consisting only of”.

1. A filler for an anaesthetic vaporizer used for veterinary surgery, said filler attached to a cylindrical bucket portion of said vaporizer, wherein said filler is curved in shape and having a concave mounting surface that is obverse to said cylindrical bucket portion, said filler having fluid conduits therein interconnecting first apertures on said concave mounting concave surface and a filler-port unit, said first apertures on said mounting surface aligning with associated second apertures on said bucket, and a gasket with a plurality of first holes aligned with first and second apertures is disposed between said bucket and said filler.

2. A filler as claimed in claim 1, wherein said filler is provided with a plurality of third apertures, aligned with a plurality of fourth apertures in said bucket and a plurality of second holes in said gasket, said third plurality of apertures allowing for threaded fasteners to be inserted from external of said filler through said plurality of second holes in said gasket and through to said fourth apertures in said bucket to attach said filler to said bucket.

3. A filler as claimed in claim 1, wherein said threaded fasteners are captive screws that do not extend into the reservoir disposed within said cylindrical bucket.

4. A filler as claimed in claim 1, wherein said filler has a sight window disposed thereon at or near one end of said filler, and said opposed end has an upper portion overhanging a side mounting surface to which a filler port unit is attached, said filler port unit having a lower surface that is substantially flush with the bottom surface of said filler.

5. A filler as claimed in claim 1, wherein the fluidally connected breather holes in said filler are spaced both vertically and horizontally to each other.

6. A filler as claimed in claim 1, wherein the body of said filler is made of brass and coated with nickel.

7. A filler as claimed in claim 1, wherein said vaporizer is for vaporising anaesthetic liquid being dispensed to an animal patient via an associated anaesthetic delivery machine.

8. A vaporizer and filler in combination, said vaporizer having a cylindrical outer bucket containing a reservoir for containing anaesthetic, said filler having a concave mounting surface obverse to said outer cylindrical bucket, said filler mounted to said vaporizer via said concave mounting surface abutting against said cylindrical outer bucket with a gasket disposed there between, said filler attached to said vaporizer by captive screws fitted from external of said vaporizer.

9. A vaporizer and filler in combination as claimed in claim 8, wherein said filler has a filler-port unit attached thereto at or near a lower corner portion of said filler, and said filler-port unit having a lower surface that is substantially flush with the bottom surface of said filler.

10. A vaporizer and filler in combination as claimed in claim 8, wherein said filler has fluidally connected breather holes therein spaced both vertically and horizontally to each other.
